(19) United States
(12) Patent Application Publication
(10) Pub. No.: US 2019/0380677 A1
(43) Pub. Date: Dec. 19, 2019

(54) METHOD AND APPARATUS FOR ULTRASONIC CONTINUOUS MEASUREMENT OF BLOOD VESSEL DIAMETER

(71) Applicants: Yuu Ono, Nepean (CA); Masayoshi Yoshida, Ottawa (CA); Andy Huang, Ottawa (CA); Sreeraman Rajan, Kanata (CA)

(72) Inventors: Yuu Ono, Nepean (CA); Masayoshi Yoshida, Ottawa (CA); Andy Huang, Ottawa (CA); Sreeraman Rajan, Kanata (CA)

(21) Appl. No.: 16/439,850

(22) Filed: Jun. 13, 2019

Related U.S. Application Data
(60) Provisional application No. 62/685,334, filed on Jun. 15, 2018.

(51) Int. Cl. A61B 8/08; A61B 8/00

(52) U.S. Cl. CPC A61B 8/0891; A61B 8/5223; A61B 8/4281; A61B 8/4455

(57) ABSTRACT

Changes in the mechanical and/or physiological properties of blood vessel serve as important indicators for prediction of cardiovascular disease. One challenge of the ultrasonic blood vessel measurement is the compression of the vessel due to the weight of a conventional ultrasound probe employed, resulting in the error on the diameter estimation. The presented invention addresses this issue with low-cost wearable ultrasound film sensors, which is flexible and lightweight so that it does not deform the blood vessel of interest beneath the sensor or restrict its motion during the measurement. In the preferred embodiment, the sensor is attached on a body surface above a blood vessel of interest. Pulsed ultrasound is transmitted into the body and the echoes from the posterior and anterior boundaries of a blood vessel are acquired with an ultrasound M-mode measurement. Then, the depth of each boundary is obtained with an ultrasound time of light technique. Finally, the blood vessel diameter is calculated by subtracting the depth of posterior boundary from that of anterior boundary of the blood vessel.

### BACKGROUND

### Field of the Invention

The present invention relates to an apparatus and method for continuous measurement of blood vessel diameter using a single or multiple wearable ultrasonic sensors.

### Description of the Related Art

Monitoring of the blood vessel diameter can be used for diagnosis of diseases. This diameter can be used directly to derive properties, such as blood vessel stiffness and blood pressure, that can serve as an indicator for cardiovascular state and function. One such indicator is endothelial dysfunction, which is known to play a role in development of atherosclerosis and cardiovascular disease.

A variety of different imaging modalities and techniques are currently available for measurement of blood vessel diameter. For measurement of endothelial dysfunction one of the most popular approaches is the measurement of change in arterial diameter through ultrasound imaging during flow mediated dilation. Blood pressure could be also obtained from the measurements of arterial diameter changes and pulse wave velocity.

One challenge that is encountered during blood vessel diameter measurement using conventional clinical ultrasonic probes is the motion artifacts. Due to the size and weight of handheld ultrasonic probes, it is difficult to have the reliable and/or consistent contact of the handheld probe onto the desired measurement area. This can result in motion artifacts due to inconsistent probe motion, deformation of the blood vessel due to the probe weight, and shifts in the measurement area during the data acquisition. In addition, unintended body motion of a patient can also contribute to motion artifacts. Furthermore, since the clinical probe is attached to the skin surface during the measurement, the weight and pressure of the clinical probe applied on the skin surface may restrict motion of the underlying tissue, which could lead to inaccurate measurements of tissue displacement at the location of interest. These factors make it difficult to maintain a consistent and accurate measurement of blood vessel diameter over time.

### SUMMARY

The present invention relates to an apparatus and method for continuous measurement of the blood vessel diameter of a patient. The resultant diameter measurements are displayed, in real time, to provide a continuous, blood vessel diameter measurement.

### BRIEF DESCRIPTION OF THE DRAWINGS

Key features of the invention and performance may be referenced in the appended figures. Although the preferred embodiment is demonstrated in these figures it is not considered as limiting its scope with regards to other potential embodiments of the invention.

FIG. 1 depicts a schematic of the sensor structure. Each particular embodiment of the invention is constructed from a polymer piezoelectric film having a top and bottom electrodes. The ultrasonic active sensing area is determined by the overlapping areas of the top and bottom electrodes. The sensing area is selected to be large enough to cover the blood vessel of interest even with slight lateral motion shifts of the blood vessel during the measurements. The sensor is covered by an acoustic insulator and electromagnetic shielding layer for acoustical and electrical noise shielding, respectively. The most outer layer is a protection layer using non-conductive polymer film for sensor protection, electrical insulation and waterproofing. The sensor is flexible and lightweight so that it can be attached onto a curved and deformable body surface without deforming the underlying soft tissues including blood vessels or restricting the underlying tissue motion.

FIG. 2 illustrates one embodiment of the invention for measurement of blood vessel diameter. As shown, the one or more of the embodiments are secured over the blood vessel of interest. Each embodiment is driven by an ultrasonic pulser/receiver. The signal received by the pulser/receiver is sent to a data acquisition system for calculation of the blood vessel diameter.

The embodiment is secured over the blood vessel of a subject with a layer of ultrasound coupling material between the sensor and skin surface. An adhesive tape may be applied over the wearable sensor to secure it above the blood vessel. An ultrasound imaging device may be used to identify the location of the blood vessel of interest and ensure a proper sensor placement. Additional embodiments can be attached adjacent to the previous sensor for measurement with multiple sensors.

Pulsed ultrasound is transmitted into the subject's body by the embodiment. At the blood vessel boundaries, echoes are produced due to the difference in acoustic properties between the blood vessel and surrounding tissue. The system measures the echoes that resulted from their transmitted ultrasound to record the ultrasound radio frequency (RF) signals.

FIG. 3 illustrates the ultrasound RF signals, where the signals A and B indicate the boundary echoes from the anterior and posterior sides of the blood vessel, respectively. The depths and their changes of the anterior and posterior sides of blood vessel boundaries can be calculated by analyzing the time delay of ultrasonic RF signals acquired using ultrasonic time-of-flight method, and the blood vessel diameter can be obtained by subtracting the depth of the anterior boundary from that of the posterior boundary of the blood vessel. The calculated diameter from additional sensors are collated or appropriately fused to improve measurement accuracy.

FIG. 4. illustrates the system model for blood vessel diameter measurement using the wearable ultrasonic sensor. Ultrasonic measurements from the sensor are obtained in M-mode. The signals are then analyzed, and the anterior and posterior boundary motions are obtained. Subtraction of the depth of the posterior boundary from the anterior boundary is used to determine the boundary diameter.

FIG. 5 illustrates the general flow for processing of the ultrasonic signals acquired from the wearable ultrasonic sensor. The ultrasound signal is first filtered to reduce noise from the signal and reduce errors. The locations of the anterior and posterior boundary of the blood vessel of interest are determined. Finally, the motion of the determined boundary is measured.

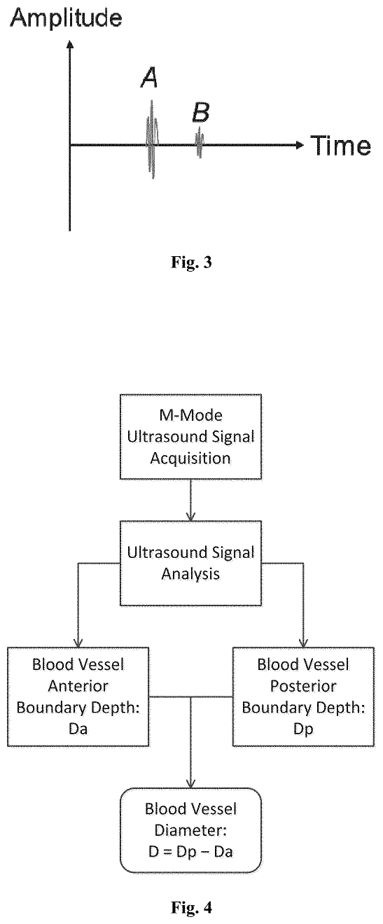

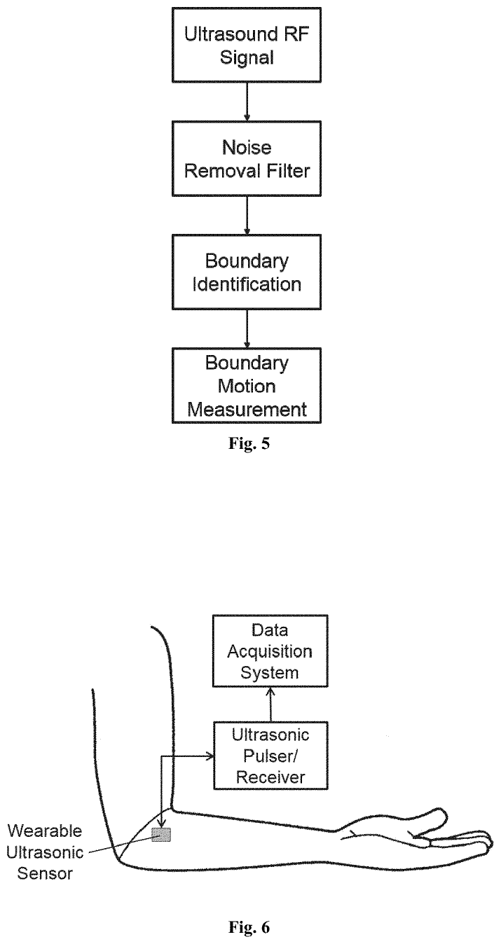

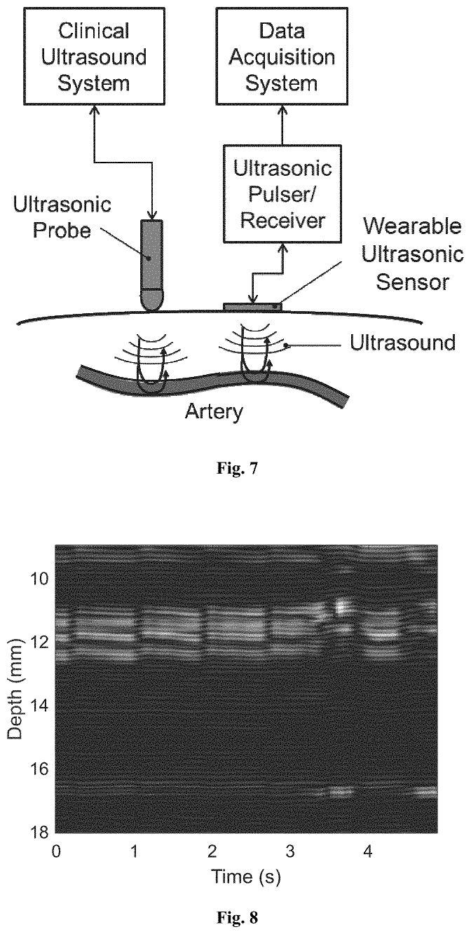

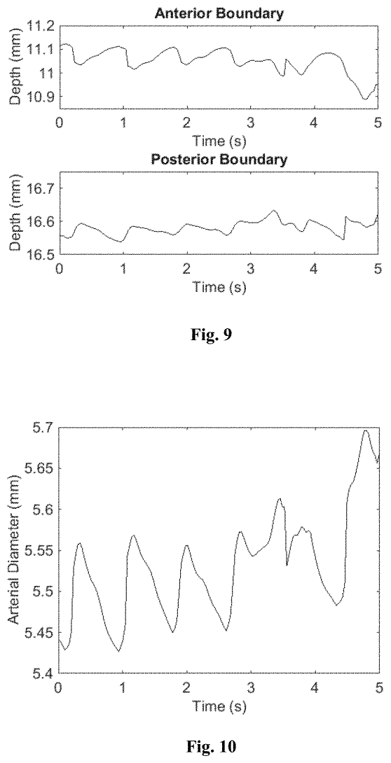

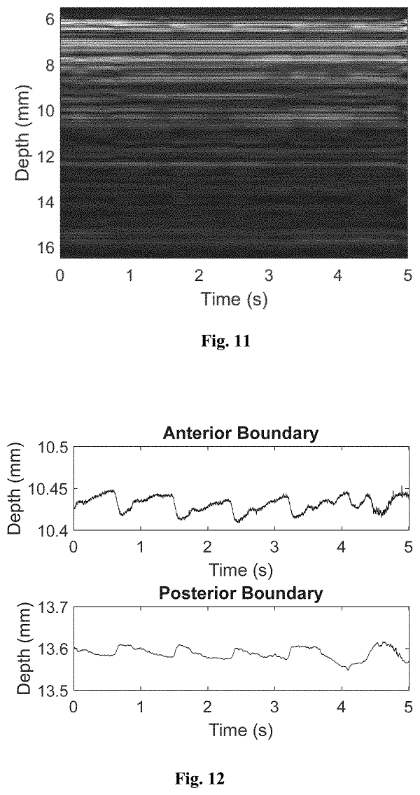

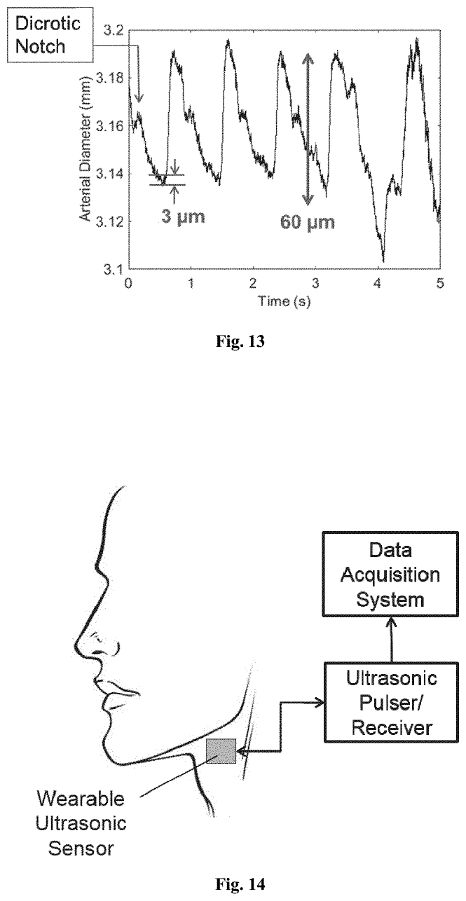

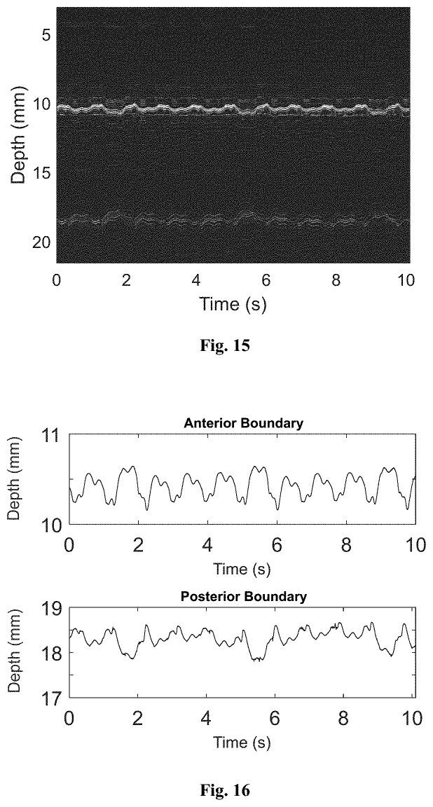

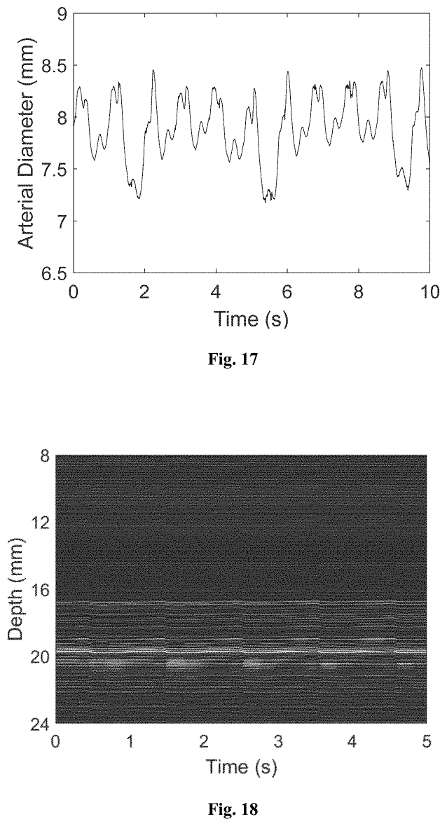

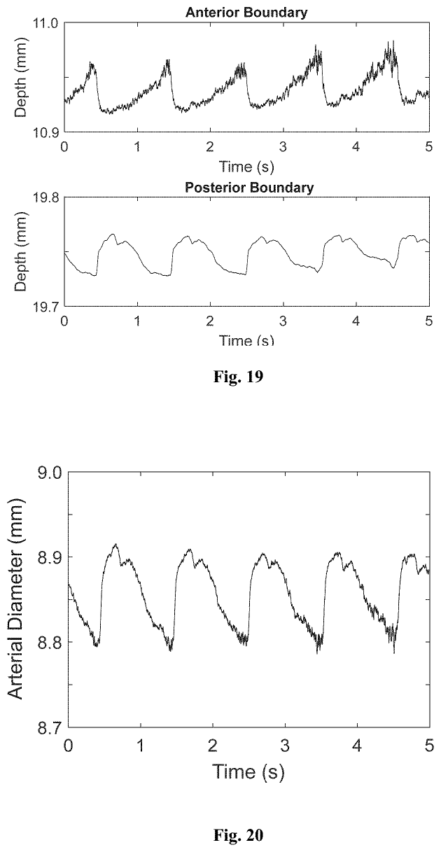

FIG. 6 illustrates one embodiment of the invention for measurement of brachial artery. The ultrasonic sensor placed over the brachial artery and is driven by a system as shown in FIG. 2.

FIG. 7 illustrates one embodiment of simultaneous measurement of the brachial artery using the invention alongside a clinical ultrasound system. The clinical ultrasound probe is placed beside the invention to obtain simultaneous measurement of the artery of interest. The invention is connected to a system as described in FIG. 2.

FIG. 8 presents an example of an ultrasound M-mode measurement obtained with a healthy male subject, using a clinical ultrasound system. The sensor was attached on the left arm above a brachia artery as shown in FIG. 6.

FIG. 9 presents a measurement example of the depths of the anterior and posterior sides of the brachia artery of a healthy male subject, using a clinical ultrasound system. The depths varied due to heartbeats.

The resulting blood vessel diameter obtained by the difference between the depths of the anterior and posterior sides of artery is shown in FIG. 10.

FIG. 10 presents the diameter of the brachial artery obtained from the results in FIG. 9. The result indicates a heart rate of 1.2 Hz over the first four cycles, corresponding to a heart rate of 72 bpm. From the results obtained over the first 4 cardiac cycles, a mean arterial diameter of 5.44 mm and 5.56 mm during diastolic and systolic phases of the cardiac cycle and a mean increase in arterial diameter of 0.12 mm from diastolic to systolic states over the cardiac cycle. In the last two seconds of the measurement from 3 s to 5 s, the result of the arterial diameter was not consistent, probably due to the motion artifact caused by the ultrasonic probe and/or the subject's arm movement.

FIG. 11 presents an example of an ultrasound M-mode measurement obtained with a healthy male subject, using a wearable ultrasonic sensor. The sensor was attached on the left arm above a brachia artery as shown in FIG. 6.

FIG. 12 presents a measurement example of the depths of the anterior and posterior sides of the brachia artery of a healthy male subject, using a wearable ultrasonic sensor. The depths varied due to heartbeats.

The resulting blood vessel diameter obtained by the difference between the depths of the anterior and posterior sides of artery is shown in FIG. 10.

FIG. 13 presents the diameter of the brachial artery obtained from the results in FIG. 9. The result indicates a heart rate of 1.15 Hz over the first four cycles, corresponding to a heart rate of 69 bpm. In addition to the increase and decrease in the arterial diameter as the cardiac cycle transitions between the systolic and diastolic phase, the wearable ultrasound sensor was able to also detect the dicrotic notch corresponding to the closing of the aortic valve. From the results obtained over the first 4 cardiac cycles, a mean arterial diameter of 3.13 mm and 3.19 mm at diastolic and systolic pressure. The embodiment was able to capture a 60 μm change in arterial diameter over the cardiac cycle.

FIG. 14 illustrates one embodiment of the invention for measurement of carotid artery. The wearable ultrasonic sensor placed over the carotid artery and is driven by a system as shown in FIG. 2.

FIG. 15 presents an example of an ultrasound M-mode measurement of the carotid artery obtained with a healthy male subject, using a wearable ultrasonic sensor. The sensor was attached on the throat above the carotid artery as shown in FIG. 14.

FIG. 16 presents a measurement example of the depths of the anterior and posterior sides of the carotid artery of a healthy male subject, using a wearable ultrasonic sensor. The depths varied due to heartbeats. The resulting blood vessel diameter obtained by the difference between the depths of the anterior and posterior sides of artery is shown in FIG. 17.

FIG. 17 presents the diameter of the carotid artery obtained from the results in FIG. 16. The result indicates a heart rate of 1.1 Hz over the first ten cycles, corresponding to a heart rate of 66 bpm. In addition to the motion of the carotid artery, motion artifacts are present due to motion of the vein and breathing. From the results obtained over the first 10 cardiac cycles, a mean arterial diameter of 7.92 mm and 8.66 mm at diastolic and systolic pressure, respectively. The embodiment was able to capture a 0.74 mm change in arterial diameter over the cardiac cycle.

FIG. 18 presents an example of an ultrasound M-mode measurement of the carotid artery obtained with a healthy male subject with held breath, using a wearable ultrasonic sensor. The sensor was attached on the throat above the carotid artery as shown in FIG. 14.

FIG. 19 presents a measurement example of the depths of the anterior and posterior sides of the carotid artery of a healthy male subject, using a wearable ultrasonic sensor. The depths varied due to heartbeats.

The resulting blood vessel diameter obtained by the difference between the depths of the anterior and posterior sides of artery is shown in FIG. 20.

FIG. 20 presents the diameter of the carotid artery obtained from the results in FIG. 19. The result indicates a heart rate of 1.1 Hz over the first ten cycles, corresponding to a heart rate of 66 bpm. From the results obtained over the first 10 cardiac cycles, a mean arterial diameter of 8.79 mm and 8.91 mm at diastolic and systolic pressure, respectively. The embodiment was able to capture a 0.12 mm change in arterial diameter over the cardiac cycle.

### DETAILED DESCRIPTION

### I. Introduction

Monitoring of the physical properties of tissue has a wide range of clinical applications for diagnosis of diseases. Measurement of physical properties, such as arterial stiffness, can serve as an indicator for cardiovascular state and function [1-3]. Endothelial dysfunction is a precursor to atherosclerosis and cardiovascular diseases. Monitoring for endothelial function may lead to earlier prevention of cardiovascular diseases. Generally, imaging modalities are utilized for such monitoring purposes.

A variety of different imaging modalities and techniques are available for measurement of different arterial properties [3-5]. For measurement of endothelial dysfunction, one of the most popular approaches is the continuous measurement of change in arterial diameter through ultrasound imaging during flow mediated dilation [6, 7]. Blood pressure measurement is a standard and common technique to diagnose a patient health conditions. It has been demonstrated that blood pressure can be measured from the vessel diameter changes and the pulse wave velocity [8]. The stiffness of the blood vessel could be also evaluated from the vessel diameter changes and blood pressure measured.

A major challenge that is encountered during arterial diameter measurement using conventional clinical ultrasonic probes is the motion artifact and the deformation of the underlying tissues including the blood vessels of interest. Due to the size and weight of handheld ultrasonic probes, it is difficult to secure the probe onto the desired measurement area. This can result in motion artifact and shifts in the measurement area during the data acquisition. Unintended movement of the subject can also contribute to additional motion. Furthermore, since the clinical probe is attached to the skin surface during the measurement, the weight and pressure of the clinical probe applied on the skin surface may deform the underlying tissues and/or restrict their motion, which could lead to inaccurate measurements of tissue displacement at the location of interest. These motion artifacts and tissues deformations make it difficult to maintain a consistent measurement over time.

As a solution to the above issues, this patent application proposes the use of a lightweight, wearable, and flexible ultrasonic sensor proposed in [9, 10] to measure arterial diameter. This sensor can be secured over the area of interest, and can ensure a consistent measurement area during continuous measurements. The lightweight and flexible nature of the sensor helps to reduce or avoid restriction to the arterial motion of interest under it.

### II. Measurement Methods of Blood Vessel Diameter

### A. Measurement Setup

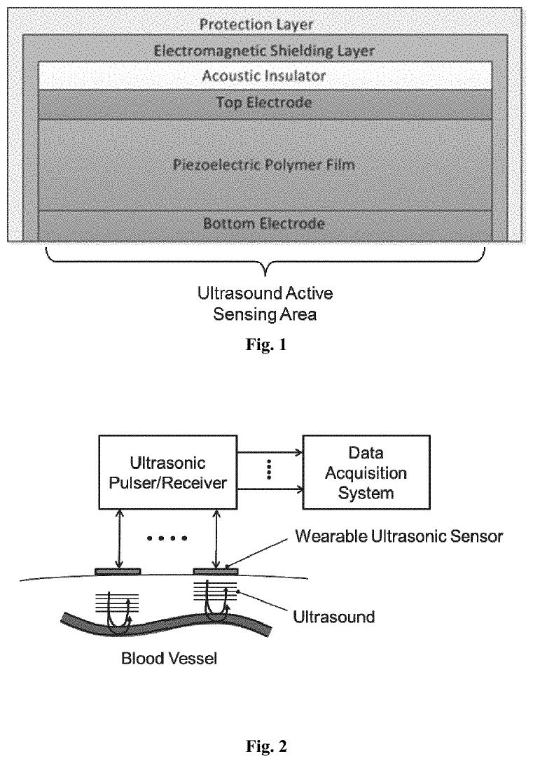

FIG. 1 depicts a schematic of the wearable ultrasonic sensor structure. Each of this particular embodiment of the invention is constructed from a polymer piezoelectric film having a top and bottom electrode. The ultrasonic active sensing area is determined by the overlapping area of the top and bottom electrodes. The sensing area is selected to be large enough to cover the blood vessel of interest even with slight lateral motion shifts of the blood vessel during the measurements. The sensor is covered by an acoustic insulator and electromagnetic shielding layer for acoustical and electrical noise shielding, respectively. The most outer layer is a protection layer that uses non-conductive polymer films for sensor protection, electrical insulation and waterproof. The sensor is flexible and lightweight so that it can be attached onto a curved and deformable body surface without deforming underlying soft tissues including blood vessels or restricting the underlying tissue motion.

The wearable ultrasonic sensor was constructed from polymeric piezoelectric film [9, 10]. As an example, the sensor was constructed with 110-μm thick polyvinylidene difluoride (PVDF) piezoelectric film. It had an active ultrasonic area (electrode size) of 15 mm by 15 mm, and the total thickness and the weight of the sensor were 0.2 mm and less than 1 g, respectively [9]. Such a sensor can be also constructed with other types of polymeric piezoelectric films such as nylon-11, polyvinyl fluoride, polyvinyl chloride, polyurea, vinylidene fluoride-trifluoro ethylene copolymer (P(VDF-TrFE)), and polylactic acid (PLA), for instance. The polymeric film may be a single layer or multiple layers. The thickness and the number of the polymeric piezoelectric layers are chosen based on the desired ultrasound characteristics for applications such as operating ultrasound frequency and frequency bandwidth. The wearable sensor can be attached onto a curved and deformable body surface without restricting the underlying tissue motion.

The system configuration for ultrasound measurement using the wearable ultrasonic sensor is shown in FIG. 2. As shown, the one or more of the wearable ultrasonic sensors are secured over the blood vessel of interest. The wearable ultrasonic sensors are attached on the skin surface of a subject with a layer of ultrasonic couplant material between the sensor and skin surface. The ultrasound couplant material could be liquids, gel, adhesives, glues, adhesive tape, or double-sided adhesive film, for instance. Each embodiment is driven by an ultrasonic pulser/receiver. The signal received by the pulser/receiver is sent to a data acquisition system for calculation of the blood vessel diameter.

In this study, the wearable ultrasonic sensor was driven by an ultrasonic pulser/receiver (Model 5900PR, Olympus Panametrics NDT, Waltham, Mass.) to transmit and receive ultrasonic radio frequency (RF) signals. The received ultrasonic RF signals were digitized by an A/D converter (Model ATS 9440, Alazartech, Montreal, QC) controlled by a PC. The ultrasonic measurements were performed in M-mode with a frame rate of 1 kHz and a sampling rate of 125 MHz.

Ultrasonic pulse echo measurements were performed to measure the change in arterial diameter due to heart beats of a subject at rest. Based on time-of-flight information obtained from received ultrasound echoes the motion of arterial boundaries may be captured as shown in FIG. 3. FIG. 3 illustrates the ultrasound RF signals, where the signals A and B indicate the boundary echoes from the anterior and posterior sides of the blood vessel, respectively. By means of an ultrasonic time-of-flight method, the depths and their changes of the anterior and posterior sides of blood vessel boundaries can be calculated by analyzing the time delay of the acquired ultrasonic RF signals, and thus the blood vessel diameter can be obtained by subtracting the depth of the anterior boundary from that of the posterior boundary of the blood vessel. The calculated diameter from additional sensors may be fused to improve measurement accuracy.

The overall general system model for measurement for blood vessel diameter is shown in FIG. 4. The signals acquired from the wearable ultrasonic sensor are analyzed (as described in the next section) and then the depths of the anterior and posterior depths are determined. The blood vessel diameter is then calculated to be the difference in depth between the posterior and anterior boundaries.

### B. Ultrasonic Signal Analysis

The general ultrasonic signal analysis performed on the signals acquired by the wearable ultrasonic sensor is shown in FIG. 5. The ultrasonic RF signal obtained by the wearable ultrasonic sensor were first processed to reduce the signal noise. A variety of approaches are available for random noise removal, such as moving average filter, low and bandpass filters, and adaptive filters. Due to the weaker signal strength of the wearable ultrasonic sensor, the speckle noise may mask the deeper boundary echoes from the posterior boundary within the received ultrasonic RF signals. Therefore, in addition to the moving average filter, a speckle removal filter was required for the signals acquired by the wearable ultrasonic sensor system. A stationary wavelet filter has been shown to reduce speckle noise on a simulated ultrasound signal [11]. In this paper, a wavelet filter using a Daubechies 4 wavelet at a decomposition level of 7 was applied to reduce speckle noise.

After the signal has been filtered, the location of the blood vessel boundaries can be determined. There were two general approaches that were utilized the identify the location of the boundary motion. The first approach is to use the ultrasound B-mode image to identify expected location of artery and utilize ultrasound peak echoes at that region. A conventional medical ultrasound imaging system may be used to identify the initial locations of the anterior and posterior boundaries of a blood vessel of interest. The second approach is to examine the motion present at each depth and select the blood vessel location based on measured motion.

In the peak location approach for boundary detection, the approximate location of the blood vessel boundary were first located based on B-mode images used to identify the location of the blood vessel during sensor placement. The peak echoes located at the depths close to the identified boundary from the B-mode images were selected as the location of the blood vessel boundary.

The second approach utilized the measured motion at the boundary. Similar to the peak detection method, the approximate location of the blood vessel boundaries were first located based on the B-mode images. Then, the location of the anterior boundary was first determined by measuring the motion at different depths around the approximate estimated location of the anterior boundary. The depth with the maximum motion was then selected as the location of the anterior boundary. This was repeated for the location of the posterior boundary. However, in addition to searching for depth with the maximum motion, the depth also required to demonstrate posterior boundary moving in opposite direction of the anterior boundary. The expansion of the blood vessel should result in motion of the anterior and posterior boundary in opposite directions; therefore the posterior boundary's motion must demonstrate a motion in opposite direction to the anterior boundary's motion.

Due to the relative small motion of the boundary motions, the motion at each boundary was determined using a phase-tracking method [12]. Finally, the blood vessel diameter was calculated from the difference of the boundaries' depths.

Various embodiments of the device include variations in shape, material composition, and construction methods for the sensor and control systems. Variation of the embodiments may also allow for variations in number of sensors and combinations of measured ultrasound signal.

### III. Measurement Examples

The capability of the wearable ultrasonic sensor was investigated based on measurement of two arteries. The two arteries of interest were the brachial and carotid arteries. All measurements were performed on a healthy male subject. The measurement of the brachial and carotid arteries are described as follows.

### A. Brachial Artery

1) Measurement Setup

The measurement setup of the wearable ultrasonic sensor for measurement of the brachial artery diameter used in this study is shown in FIG. 6. The wearable ultrasonic sensor had an active ultrasonic area (electrode size) of 20 mm by 20 mm. In addition to the measurement using the wearable ultrasonic sensor measurements, a clinical ultrasonic imaging system (Model: PICUS, ESAOTE, Maastricht, Netherlands) were used for a comparison purpose. The measurements from the clinical ultrasound system were performed with a linear ultrasonic probe (Model: L10-5) in B-mode with a frame rate of 30 Hz and a sampling rate of 33.3 MHz. The measurement setup for synchronous measurement with the clinical ultrasound system and wearable ultrasonic sensor is shown in FIG. 7. However, due to hardware restrictions, it was not possible to synchronize the two systems perfectly. Synchronization with an almost time difference of a second was achieved.

The clinical ultrasonic probe and wearable ultrasonic sensor were secured over the brachial artery of a male subject's left arm with a layer of couplant gel between the probe/sensor and skin surface, as shown in FIG. 7. A measurement stand was used to hold the clinical ultrasonic probe and an adhesive tape was applied over the wearable ultrasonic sensor to secure them above the brachial artery. The arm of the subject was resting on a table and care was taken to keep the arm stationary while 5 s of measurements were collected. The clinical ultrasound system performed the measurement in B-mode to ensure detection of the brachial artery. Due to the single transducer configuration of the wearable ultrasonic sensor, measurements with the wearable ultrasound sensor system were acquired in M-mode. The size of the wearable ultrasonic sensor was selected large enough to ensure detection of the brachial artery.

The respective systems measured the echoes that resulted from their transmitted ultrasound. Based on the time-of-flight (time delay) of the boundary echoes obtained by analyzing the ultrasonic RF signals acquired, the depths of the anterior and posterior sides of arterial boundaries were measured, and then the arterial diameter was obtained.

2) Clinical Ultrasound Probe Results

The M-mode measurements derived from the clinical ultrasound B-mode measurements are shown in FIG. 8. The motions of the anterior and posterior boundaries of the brachial artery measured by the clinical ultrasonic probe are shown in FIG. 9, and the resulting arterial diameter is shown in FIG. 10. We observed the 1.20 Hz cyclical motion of the arterial walls, corresponding to the subject's heart rate of 72 bpm, obtained from the three cycles between 0.1 s and 2.6 s. The result indicates the expansion of the artery, signified by the steep negative motion of the anterior boundary (towards the probe) and positive motion at the posterior boundary (away from the probe). These motions of the arterial boundaries correspond to the phases in the cardiac cycle and variation in the blood pressure. The maximum arterial diameter corresponds to the systolic pressure, while the minimum arterial diameter corresponds to the diastolic blood pressure. Based on the measured arterial diameter presented in FIG. 10, we calculated the mean diameter of 5.44 mm and 5.56 mm during diastolic and systolic phases of the cardiac cycle and a mean increase in arterial diameter of 0.12 mm from diastolic to systolic state over the cardiac cycle. In the last two seconds of the measurement from 3 s to 5 s, the result of the arterial diameter was not consistent, probably due to the motion artifact caused by the ultrasonic probe and/or the subject's arm movement.

3) Wearable Ultrasonic Sensor Results

The M-mode measurements obtained by the wearable ultrasonic sensor are shown in FIG. 11. The results of arterial boundary motion obtained by the proposed wearable ultrasonic sensor and the resulting arterial diameter are shown in FIG. 12 and FIG. 13, respectively. The results in FIG. 13 indicated a heart rate of 1.15 Hz over the first four cycles, corresponding to a heart rate of 69 bpm, which has a reasonable agreement with the heart rate obtained by the clinical ultrasound probe. In addition to the increase and decrease in the arterial diameter as the cardiac cycle transitions between the systolic and diastolic phase, the dicrotic notches corresponding to the closing of the aortic valve were observed. From the results obtained over the first four cardiac cycles, a mean arterial diameter of 3.13 mm and 3.19 mm at diastolic and systolic pressure, respectively. The wearable sensor was able to capture a 0.06 mm change in arterial diameter over the cardiac cycle.

4) Discussions

Both the clinical ultrasound probe and the wearable ultrasound sensor demonstrated the capability to measure the change in diameter of the brachial artery during cardiac cycles. In the results obtained by the respective systems, the change in arterial diameter between the systolic to diastolic phases was captured and similar trends in arterial diameters over the cardiac cycle were observed.

The mean arterial diameter at diastolic and systolic pressure measured by the clinical ultrasound probe was 5.44 mm and 5.56 mm, respectively. The proposed wearable ultrasound sensor measured a corresponding mean arterial diameter of 3.13 mm and 3.19 mm at diastolic and systolic pressures, respectively, or approximately 57% of the diameter measured by the clinical probe. This difference in diameter could be attributed to several factors. One factor is that the artery might not be parallel to the skin surface, which may have resulted in different alignments of the clinical probe and wearable sensor with respect to the artery. If the transmitted ultrasound was not normal to the artery, the estimated diameters could be different from the true diameter. Another factor of error is the identification of the boundary locations in the measured ultrasound RF signals.

One of the challenges faced by the proposed wearable sensor was the relatively low ultrasound signal strength compared to the clinical ultrasound probe. This resulted in higher measurement noise and lower boundary echo amplitude. While this was not a significant issue for the anterior boundary, the posterior boundary echo was more challenging to locate as it was located deeper (and therefore higher ultrasound signal attenuation). This resulted in a boundary echo closer to the noise floor and therefore was difficult to identify. The precise location of the posterior boundary is an ongoing research topic.

Comparatively, the stronger ultrasound produced by the clinical ultrasound probe alongside B-mode measurement made the larger boundary echoes easier to identify. However, arterial diameter measurement with the clinical ultrasound probe faced challenges due to undesired motion of the ultrasonic probe and/or the subject during the measurements. Such errors can be observed in FIG. 4 after 3 s, where subject motion probably resulted in loss of clear cardiac pattern compared to the time from 0 s to 3 s. This sensitivity to motion limits the potential applications of clinical ultrasound system for continuous arterial measurement.

### B. Carotid Artery

1) Measurement Setup

The measurement setup for the measurement of the carotid artery using the wearable ultrasound sensor is shown FIG. 14. The wearable ultrasonic sensor had an active ultrasonic area (electrode size) of 20 mm by 20 mm. The measurements were performed on a healthy male subject. The subject was lying on an inclined bed surface. A clinical ultrasound probe was used to locate the carotid artery using B-mode measurements. The wearable ultrasonic sensor was placed over the carotid artery with a layer of couplant gel between the sensor and skin surface. Due to the single transducer configuration of the wearable ultrasonic sensor, measurements with the wearable ultrasound sensor system were acquired in M-mode. The size of the wearable ultrasonic sensor was selected large enough to ensure detection of the carotid artery.

The subject was first asked to breathe normally and then 10 s of measurements were recorded using the wearable ultrasound sensor. The measurements were performed at an M-mode frame rate of 1 kHz and a sample rate of 125 MHz. Another set of measurements were conducted where the subject was asked to stop breathing for 5 s while the measurements were obtained. The wearable ultrasonic sensor system measured the echoes that resulted from their transmitted ultrasound. Based on the time-of-flight (time delay) of the boundary echoes obtained by analyzing the ultrasonic RF signals acquired, the depths of the anterior and posterior sides of carotid boundaries were measured, and then the arterial diameter was obtained.

2) Wearable Ultrasonic Sensor Results

The M-mode measurement of the carotid artery obtained for the wearable ultrasonic sensor is shown in FIG. 15. The motion of the anterior and posterior boundaries of the carotid artery measured under normal breathing conditions by the wearable ultrasonic sensor are shown in FIG. 16, with the resulting arterial diameter in FIG. 17. The location of the boundaries were determined based on selection of ultrasonic echoes close to the expected carotid arterial boundaries located from B-mode measurements obtained by the clinical ultrasound system. From the figures it can be observed that the 1.1 Hz of motion corresponding to a heart rate of 66 bpm obtained from 11 cycles between 0 s to 10 s. From the results obtained over the first 10 cardiac cycles, a mean arterial diameter of 7.92 mm and 8.66 mm at diastolic and systolic pressure, respectively, were obtained. The wearable ultrasonic sensor was able to capture a 0.74 mm change in arterial diameter over the cardiac cycle.

The M-mode measurement obtained from the patient without any breathing is shown in FIG. 18. Compared to the previous results it is difficult to ascertain the location of the anterior boundary from the peak echoes. Therefore, the motion-based approach is used to identify the location of the anterior boundary, while the peak echo is selected for the posterior boundary. This results in the boundary motions shown in FIG. 19 with the resulting arterial diameter shown in FIG. 20. The results obtained from the measurement demonstrated a mean arterial diameter of 8.79 mm and 8.91 mm at diastolic and systolic pressure, respectively. This results in a measured 0.1148 mm change in arterial diameter.

3) Discussions

Similar to the results obtained for the brachial artery, the wearable ultrasonic sensor demonstrate the capability to measure the change in diameter of the carotid artery during normal cardiac cycle. The results obtained in the previous section demonstrated the ability to observe the increase and decrease in arterial diameter.

Results obtained during normal breathing shown in FIG. 17 revealed motion periodic motion artifacts. It is theorized that these artifacts are associated with motion of the jugular vein in the surrounding tissue and due to breathing motion.

Due to the posture of the patient lying on the table, the jugular vein is located right beside the brachial artery. Therefore, the motion of the vein can result in artifact motion in the boundaries of the brachial artery. This can be seen in FIG. 17 by the small spikes in the arterial diameter during the diameter increase from diastolic to systolic pressure. These small spikes are associated with the pressure wave present in the veins caused by the previous cardiac cycle.

Another source of error is the motion due to breathing, as shown by the two motion artifacts observed by the sudden large diameter changes occurring at approximately 2 s, 6 s, and 10 s. The periodic large increase in arterial diameter corresponds to the motion associated with the inhalation and exhalation during breathing.

The periodic motion due to breathing is verified based on measurements obtained while the subject held their breath to remove breathing motion artifacts. Based on the results shown in FIG. 20, the arterial diameter measurement demonstrated less variation and no periodic variations in diameter besides the 1 Hz motion.

Another difference between the results obtained with normal breathing and hold breath are the difference in diameter amplitude and range of motion. The sensor was moved between measurements and therefore the exact measurement area was different between the two measurements. This resulted in measurement of different sections of the carotid artery. In addition, the carotid artery does not lie exactly parallel to the skin surface, and therefore change in measurement position of the sensor can result in different alignment in measurement of the artery. This can also result in the difference in observed motion between the diameter motion in FIG. 17 and FIG. 20.

### REFERENCES

[1] M. C. Corretti, T. J. Anderson, E. J. Benjamin, D. Celermajer, F. Charbonneau, M. A. Creager, J. Deanfield, H. Drexler, M. Gerhard-Herman, D. Herrington, P. Vallance, J. Vita, and R. Vogel, “Guidelines for the ultrasound assessment of endothelial-dependent flow-mediated vasodilation of the brachial artery,” J. Amer. Coll. Cardiol., vol. 39, no. 2, pp. 257-265, 2002.
[2] H. Tomiyama and A. Yamashina, “Non-Invasive Vascular Function Tests: Their Pathophysiological Background and Clinical Application,” Circulation J., vol. 74, pp. 24-33, 2010.
[3] P. Boutouyrie, D. Fliser, D. Goldsmith, A. Covic, A. Wiecek, A. Ortiz, A. Martinez-Castelao, B. Lindholm, Z. A. Massy, G. Suleymanlar, R. Sicari, L. Gargani, G. Parati, F. Mallamaci, C. Zoccali, and G. M. London, “Assessment of arterial stiffness for clinical and epidemiological studies: methodological considerations for validation and entry into the European Renal and Cardiovascular Medicine registry,” Nephrol. Dial. Transplant, vol. 29, pp. 232-239, 2014.
[4] J. D'hooge, A. Heimdal, F. Jamal, T. Kukulski, B. Bijnens, F. Rademakers, L. Hatle, P. Suetens and G. R. Sutherland, “Regional Strain and Strain Rate Measurements by Cardiac Ultrasound: Principles, Implementation and Limitations,” European J. Echocardiography, vol. 1, pp. 154-170, 2000.
[5] J. H. Stein, C. E. Korcarz, R. T. Hurst, E. Lonn, C. B. Kendall, E. R. Mohler, S. S. Najjar, C. M. Rembold, and W. S. Post, “Use of Carotid Ultrasound to Identify Subclinical Vascular Disease and Evaluate Cardiovascular Disease Risk: A Consensus Statement from the American Society of Echocardiography Carotid Intima-Media Thickness Task Force Endorsed by the Society for Vascular Medicine,” J. Amer. Soc. Echocardiography, vol. 21, no. 2, pp. 93-111, 2008.
[6] R. A. Harris, S. K. Nishiyama, D. W. Wray and R. S. Richardson, “Ultrasound Assessment of Flow-Mediated Dilation,” Hypertension, vol. 55, pp. 075-1085, 2010.
[7] M. Kelm, “Flow-mediated dilatation in human circulation: diagnostic and therapeutic aspects,” Amer. J. Physiol. Heart Circ. Physiol., vol. 282, pp. H1-5, 2002.
[8] B. W. Beulen, N. Bijnens, G. G. Koutsouridis, P. J. Brands, M. C. Rutten and van de Vosse, Frans N, “Toward noninvasive blood pressure assessment in arteries by using ultrasound,” Ultrasound Med. Biol., vol. 37, pp. 788-797, 2011.
[9] I. AlMohimeed, H. Turkistani, and Y. Ono, “Development of wearable and flexible ultrasonic sensor for skeletal muscle monitoring,” Proc. IEEE Int. Ultrasonics Symp., pp. 1137-1140, 2013.
[10] I. AlMohimeed, M. Agarwal, and Y. Ono “Wearable Ultrasonic Sensor Using Double-Layer PVDF Films for Monitoring Tissue Motion,” Proc. IEEE Canadian Conference on Electrical and Computer Engineering (CCECE), pp. 1-4, 2018.
[11] J. L. San Emeterio, E. Pardo, and M. A. Rodriguez, “Denoising Ultrasound RF Signals by Wavelet Cycle Spinning Shrinkage,” Proc. 5^{th} European Conf Int. Federation Medical and Biological Eng., vol. 37, pp. 78-81, 2012.
[12] N. Feng, J. Zhang, and W. Wang, “A quadrature demodulation method based on tracking the ultrasound echo frequency,” Ultrasonics, vol. 44, Supplement, pp. e47-e50, 2006.

What is claimed:

1. Method and apparatus for continuous measurement of blood vessel diameter and its changes, including a wearable ultrasonic device that is made of a polymeric piezoelectric film

2. The device for claim 1, where the weight of the device is light so that it does not deform the underlying tissues or impede their motions

3. The device for claim 1, where the device is wearable and not handheld so that it does not produce motion artifacts due the sensor motion

4. The device for claim 1, where the device is flexible to ensure adequate contact with the measurement for curved and deformable surface

5. The device for claim 1, where the ultrasonic device has an ultrasonic sensing area large enough to cover the blood vessel of interest through relative lateral movement of blood vessel with regards to the measurement sensor

6. The device for claim 1, where the ultrasonic beam is large enough to cover the blood vessel of interest through relative lateral movement of blood vessel with regards to the ultrasonic beam

7. The device for claim 1, where the ultrasonic couplant material between the ultrasonic device and the skin surface of a subject could be liquids, gel, adhesives, glues, adhesive tape, or double-sided adhesive film

8. The method for claim 1, wherein calculating the blood vessel diameter includes
Measurement of the distance between the sensor and blood vessel anterior boundary (Dₐ)
Measurement of the distance between the sensor and blood vessel posterior boundary (Dₚ)
Calculating the blood vessel diameter (D=Dₚ−Dₐ)

9. The method for claim 1, comprising calculating continuous motion of blood vessel boundary during the cardiac cycle

10. The method for claim 1, co-registering the location of the sensor relative to the blood vessel

11. The method of claim 1, wherein the measurement apparatus includes an ultrasound imaging and measurement system

12. The method for claim 1, wherein calculating the pulse transit time (PTT)
Measuring the timing of blood vessel diameter change using wearable ultrasonic sensors
PPT between ECG and the wearable ultrasonic sensor
PPT between the two wearable ultrasonic sensors

13. The method of claim 1, wherein measuring the blood vessel diameter and its change during FMD (flow mediated dilation)

14. The method of claim 1, wherein measuring the PTT and blood vessel diameter for blood pressure calculation

15. The method of claim 1, wherein measuring the blood vessel diameter changes and blood pressure for the blood vessel stiffness calculation
